(19) United States
(12) Patent Application Publication
(10) Pub. No.: US 2020/0360626 A1
(43) Pub. Date: Nov. 19, 2020

(54) INTRANASAL DRUG DELIVERY DEVICE AND METHOD

(71) Applicant: Jenniss Technologies LLC, Ft. Thomas, KY (US)

(72) Inventors: Jeffrey Scott Greiwe, Fort Thomas, KY (US); Dennis Leo Breen, Fort Thomas, KY (US)

(21) Appl. No.: 16/838,705

(22) Filed: Apr. 2, 2020

Related U.S. Application Data
(60) Provisional application No. 62/832,753, filed on Apr. 11, 2019.

(51) Int. Cl. A61M 11/00; A61M 15/00

(52) U.S. Cl. CPC A61M 11/007; A61M 5/2033; A61M 15/0003

(57) ABSTRACT

An intranasal delivery device. The intranasal delivery device can have a handle and a first housing disposed in the handle. The first housing defines a first fluid chamber and an opening. A first nasal applicator member is joined to the first housing. A dispersing member is attached to the opening, the dispersing member having a first tube extending into the first fluid chamber. A valve can be in the dispersing member, the valve having a first closed state and a second open state, wherein in the first closed state the valve blocks fluid communication between the first fluid chamber and the first nasal applicator member, and in the second open state the valve facilitates fluid communication between the first fluid chamber and the first nasal applicator member. A first fluid can be in the first fluid chamber, the first fluid being a first antidote. An actuator movably connected to the handle can be movable from a first actuator position to a second actuator position to move the valve to the second open state.

### REFERENCE TO RELATED APPLICATIONS

The present application claims the priority of U.S. provisional application Ser. No. 62/832,753, entitled INTRANASAL DRUG DELIVERY DEVICE AND METHOD, filed Apr. 11, 2019, and hereby incorporates the same application herein by reference in its entirety.

### TECHNICAL FIELD

Embodiments of the technology relate, in general, to an intranasal drug delivery device and method for treating patients with a drug delivery device.

### BACKGROUND

Chemical or biological attacks on relatively large populations of people can leave large numbers of people in need of an antidote. Currently, governments and non-governmental agencies stockpile antidotes and devices for delivering antidotes. The current devices used for antidote delivery can require relatively long times to administer these antidotes to a large number of people when time to treatment is critical to minimize mass casualties.

Further, in mass casualty scenarios, there is a need to identify the patients that have receive treatment with an antidote to avoid repeated dosing/overdosing of a patient in the field at the site of the mass casualty event prior to transport, and, following ambulatory transport, to allow personnel at the healthcare facility to identify those patients who have been treated since subsequent treatments at the healthcare facility should be based on prior treatments received in the field. In attack scenarios, direct verbal or written communication between first responders and healthcare providers is not practical, therefore a way to identify those patients who have been treated and what treatment was administered is important for the welfare of the patient.

### BRIEF DESCRIPTION OF THE DRAWINGS

FIG. 1 is a schematic side elevation representation of an embodiment of an intranasal delivery device of the present disclosure.

FIG. 2 is a schematic side partial cross sectional representation of a portion of an embodiment of an intranasal delivery device of the present disclosure.

FIG. 3 is a schematic side elevation representation of an embodiment of an intranasal delivery device of the present disclosure.

FIG. 4 is a schematic side elevation representation of a portion of an embodiment of an intranasal delivery device of the present disclosure.

FIG. 5 is a schematic side elevation representation of an embodiment of an intranasal delivery device of the present disclosure.

FIG. 6 is a schematic side elevation representation of an embodiment of an intranasal delivery device of the present disclosure.

FIG. 7 is a schematic side elevation representation of an embodiment of a dispersing member of the present disclosure.

FIG. 8 is a schematic side elevation representation of an embodiment of an intranasal delivery device of the present disclosure.

FIG. 9 is a schematic front elevation representation of an embodiment of an intranasal delivery device of the present disclosure.

FIG. 10 is a schematic side elevation representation of an embodiment of an intranasal delivery device of the present disclosure.

FIG. 11 is a schematic side elevation representation of an embodiment of an intranasal delivery device of the present disclosure.

FIG. 12 is a schematic side elevation representation of an embodiment of an autoinjector device of the present disclosure.

FIG. 13 is a schematic side elevation representation of an embodiment of an intranasal delivery device of the present disclosure.

FIG. 14 is a schematic side elevation representation of an embodiment of an intranasal delivery device of the present disclosure.

FIG. 15 is a perspective representation of an embodiment of an intranasal delivery device of the present disclosure.

FIG. 16 is a perspective representation of an embodiment of an intranasal delivery device of the present disclosure.

FIG. 17 is a perspective representation of an embodiment of an intranasal delivery device of the present disclosure.

FIG. 18 is a side, partial cut away schematic representation of an embodiment of an intranasal delivery device of the present disclosure.

FIG. 19 is a perspective cut away schematic representation of an embodiment of an intranasal delivery device of the present disclosure.

### DETAILED DESCRIPTION

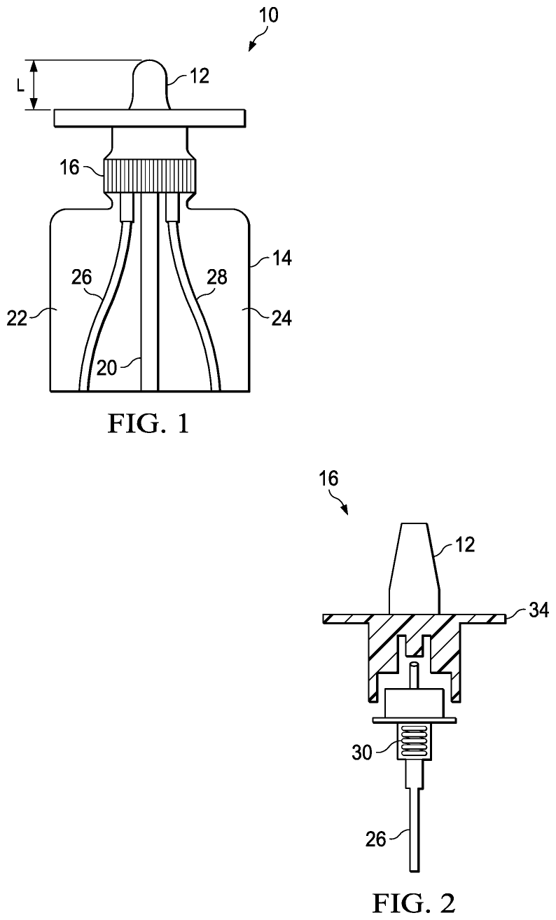

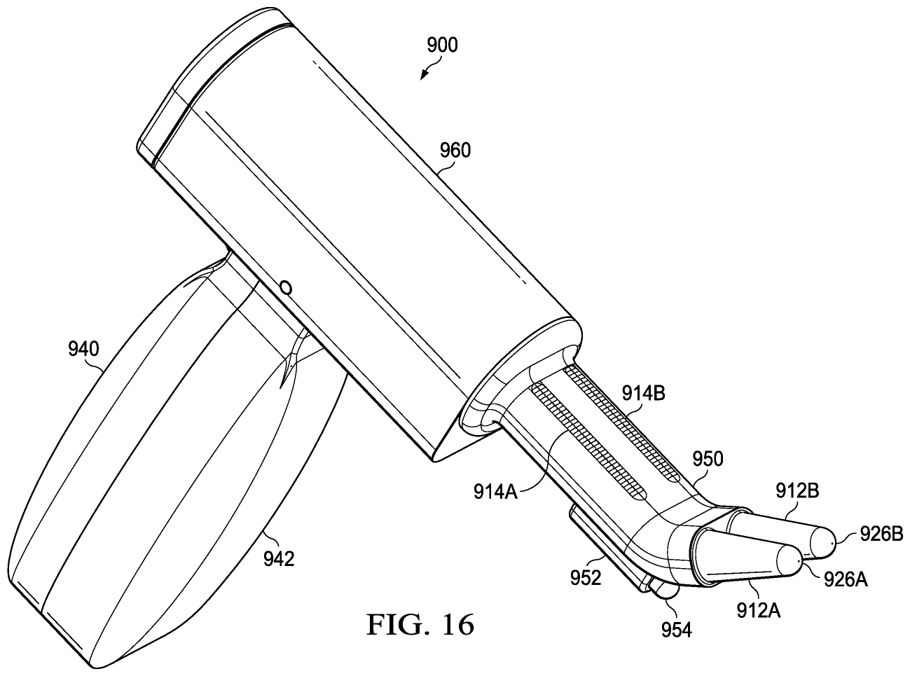

Certain embodiments are hereinafter described in detail in connection with the views and examples of FIGS. 1-19, wherein like numbers refer to like elements throughout the views.

Various non-limiting embodiments of the present disclosure will now be described to provide an overall understanding of the principles of the structure, function, and use of the apparatuses, systems, methods, and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific FIG. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Solutions to the problems associated with delivery of antidotes to persons subject to chemical or biological attack can be achieved with embodiments of the intranasal delivery device disclosed here in. The intranasal delivery device embodiments disclosed herein offer advantages in safety, ease of use, and/or speed of use, compared to, for example, autoinjectors. In general, the intranasal delivery device of the present disclosure provides a non-invasive route for rapid drug delivery directly into the nose for relatively rapid absorption through a patient's mucosal membranes. The devices and methods disclosed herein can be implemented for multi-dosing a plurality of patients in need of the dosing from a single device. That is, an intranasal delivery device can have one or more fluid chambers that can hold a quantity of fluid, such as an antidote, equivalent to from three to 100 doses of fluid. In embodiments, intranasal delivery device can deliver from five to about 25 sequential doses from one or two nasal applicator members directly to the nasal membrane of a patient. While the description herein is primarily in the context of delivering antidotes, the intranasal deliver device can be used to deliver doses of any fluid, antidote, drug, or compound.

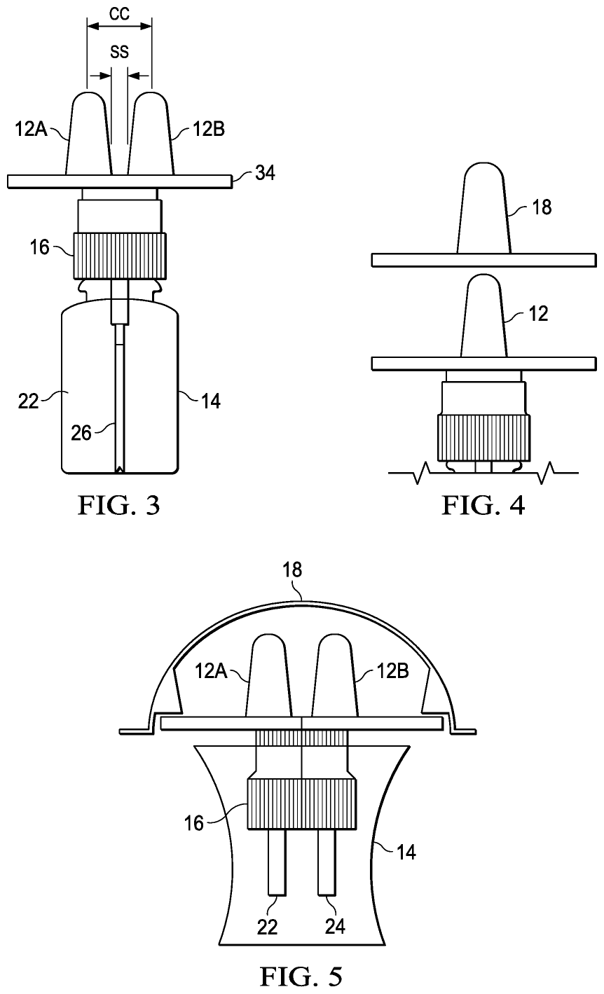

Referring now to FIG. 1, there is shown a schematic side elevation representation of an intranasal delivery device 10 for intranasal delivery of an antidote. The intranasal delivery device shown in FIG. 1 can deliver an antidote to one or both nostrils of a person in need of the antidote. That is, the intranasal delivery device 10 can deliver an antidote contained in a housing 14 through a nasal applicator member 12. A nasal applicator member as disclosed herein is intended to be sized and shaped for insertion into at least the opening of a nostril of a patient. Thus, a nasal applicator member can be considered to be a nasal insertion member. In an embodiment, the intranasal delivery device 10 can have two nasal applicator members 12 as shown and described below with respect to in FIGS. 3 and 5. Nasal applicator member(s) 12 can have defined therein a passageway (not shown) to provide fluid communication between the interior of housing 14 through a dispersing member 16 to the nostrils of a user. In an embodiment, housing 14 can be segmented internally by a divider 20 to define two discrete chambers, a first fluid chamber 22 and a second fluid chamber 24, in each of which can be contained a fluid antidote to be released through nasal applicator member 12. The fluid can be an antidote to a chemical and/or biological agent, and can be released as a mist, an atomized mist, as droplets, or combinations thereof.

In general, nasal applicator member(s) 12 can be shaped for efficient entry into the nasal passages, i.e., the nostrils, of a person in need of an antidote. A distal end of each nasal applicator member 12 can be generally rounded and convex. The material of the nasal applicator member 12 can be flexible, pliable, and sufficiently stiff as to be properly inserted into a nasal passage, i.e., a nostril. It is contemplated that the length L of nasal applicator members 12 can be from about 1 cm to about 8 cm, or from about 1 cm to about 7 cm, or from about 1 cm to about 6 cm, or from about 1 cm to about 5 cm, or from about 1 cm to about 4 cm, or from about 1 cm to about 3 cm, or from about 1 cm to about 2 cm. If two nasal applicator members 12 are used, it is contemplated that the center-to-center distance CC (FIG. 3) between the nasal applicator members 12 is suitable for the spacing between the nostrils of a patient, and may be from about 1 cm to about 8 cm, or from about 1 cm to about 7 cm, or from about 1 cm to about 6 cm, or from about 1 cm to about 5 cm, or from about 1 cm to about 4 cm, or from about 1 cm to about 3 cm, or from about 1 cm to about 2 cm. In an embodiment, two nasal applicator members 12 can have a side-to-side separation distance SS (FIG. 3) of from about 0.5 cm to about 5 cm, or from about 1 cm to about 4 cm, or from about 2 cm to about 3 cm.

The dispersing member 16, an example of which is shown in partial cutaway in FIG. 2, can be affixed, such as by threaded connection, onto a top opening of the housing 14 (FIG. 1). The dispersing member 16 can have a pressure application member 34, which can be, for example, an extension of a portion of the dispersing member 16, and upon which the fingers of a user can press down against biasing forces exerted by spring 30. When urged against the spring force of spring 30, a dose of an antidote from one or both of the first fluid chamber 22 and/or the second fluid chamber 24 can be communicated through a first tube 26 and/or a second tube 28, respectively, and released through the nasal applicator member 12 to the nostril(s) of a patient. The dispersing member 16 can include a spring-loaded valve in which a spring biases the valve in a first closed state to block fluid communication between either or both of the first fluid chamber 22 and the second fluid chamber 24 to the nasal applicator member 12, and which, upon pressure exerted against the biasing force the valve moves to a second open state that facilitates fluid communication between one or both of first fluid chamber 22 and the second fluid chamber 24 to the nasal applicator member 12. Thus, for two-nasal applicator versions, the dispersing member can include a spring-loaded valve having a first position in which fluid communication between a first and second interior chamber and first and second nasal applicators, respectively, is blocked, and a second position in which fluid communication between a first and second interior chamber and the first and second nasal applicator, respectively, is open. The valve can also be pressure activated, and could be open, for example, by the action of squeezing the housing 14, which can be a flexible, squeezable, container, to provide internal positive pressure that can open the valve and find fluid passageway to the nasal applicator member(s).

In general, the dispersing member 16 can have and utilize any of known pumps, pistons, springs, stored energy, and can operate electrically, magnetically, pneumatically or hydraulically, or with other forms of energy, as is known for nasal applicator devices.

As shown in FIG. 3, the intranasal delivery device 10 can be as described above, but can optionally have two nasal applicator members, a first nasal applicator member 12A and a second nasal applicator member 12B, such that upon use, a fluid antidote from the first fluid chamber 22 (as shown) of housing 14, or from two chambers as shown in FIG. 1, can be can be dosed through the first tube 26 or, in an embodiment, both the first tube 26 and the second tube 28, and through one or both of the first nasal applicator member 12A and the second nasal applicator member 12B, and ultimately into one or both nostrils.

As shown in FIG. 3, the intranasal delivery device 10 can be as described above, but can optionally have two nasal applicator members, a first nasal applicator member 12A and a second nasal applicator member 12B and a two discrete fluid chambers, i.e., first fluid chamber 22 and a second fluid chamber 24. First fluid chamber 22 can have a first antidote. Second fluid chamber 24 can have a second antidote, which can be the same as the first antidote or a different, second antidote. Upon use, a fluid antidote from one or both of first fluid chamber 22 and the second fluid chamber 24 of housing 14 can be can be dosed through first tube 26 and second tube 28, respectively, and through one or both of the first nasal applicator member 12A and the second nasal applicator member 12B, and ultimately into one or both nostrils. In this manner, one or two antidotes can be efficiently delivered to the nostrils of a patient with a single pump of the dispersing member 16.

As shown in FIG. 4, a safety covering 18, that can be removable, can be provided to cover the nasal applicator member 12 before use. The safety covering can be, for example, a pliable film. In an embodiment, a safety covering 18 can cover all or part of nasal applicator member 12 except for an opening where the antidote can be transferred from the intranasal delivery device 10 to the nasal cavity and which can be removed and replaced with a another covering 18 before dosing another individual. In this manner, the exchange of transmittable viruses and bacteria between individuals can be minimized or eliminated. In an embodiment, the nasal applicator member(s) can have an antimicrobial coating to minimize contamination.

As shown in the schematic representation of FIG. 5, in an embodiment, the intranasal delivery device 10 can have a first nasal applicator member 12A and a second nasal applicator member 12B, such that upon use, fluid antidotes from tube-like chambers, such as first fluid chamber 22 and second fluid chamber 24 can be can be released through both nasal applicator members, respectively, into both nostrils of a patient simultaneously. Additionally, as shown, a protective covering 18 can be provided that covers both nasal applicator members from contamination prior to use. A housing 14 can be provided that covers and protects both first fluid chamber 22 and second fluid chamber 24 from breakage and can be shaped as desired for ease of use.

In a method of use, the intranasal delivery device 10 can have contained in the first fluid chamber 22 and/or the second fluid chamber 24 a fluid antidote to a chemical or biological agent. The fluid antidote in a first fluid chamber 22 can be the same as, or different, from an antidote in a second fluid chamber 24. A first responder can carry the intranasal delivery device 10 and can dose a patient in need of a dose of an antidote to a chemical or biological agent. The first responder can remove safety covering 18, if necessary, and can insert one or both (if provided with two) nasal applicator members 12 into one or both nostrils of the patient. The first responder can then press, or otherwise activate, dispersing member 16 to urge a valve therein from a first closed state to a second, open state, to release the antidote from first fluid chamber 22 and/or second fluid chamber 24 through tube(s) 26, and/or 28 and nasal applicator member(s) 12 into the nostril(s) of the patient.

It is believed that certain benefits over the use of autoinjectors can be realized by the use of the intranasal delivery device 10 of the present disclosure. For example, the intranasal delivery device can distribute an antidote, drug, or compound to the nasal cavity to a large number of individuals in rapid sequence. The disclosed device can contain multiple doses of an antidote in a single device which can be administered to as many as 10-50 individuals. Since one device can deliver an antidote to a large number of individuals, effective of rapid delivery of the antidote can be achieved when time is of the essence, potentially saving many more lives compared to autoinjector, syringe and vials, or other dosing methods. The intranasal delivery device 10 can administer a large single dose in both nostrils simultaneously or two or more medications simultaneously with one dose.

The size, shape, and material of housing 14 can be selected for desired design and use criteria. For example, the size of housing 14 can be predetermined to be small enough to be easily shipped, stored, and carried, while being large enough to provide for multiple doses of an antidote to be delivered from a single intranasal delivery device 10. The material of housing 14 can be generally flexible so as to be squeezed a sufficient amount to provide for fluid passage through nasal applicator members 12, and rigid enough to be self-standing when not in use. The shape of housing 14 can be as desired for ease of use.

In an embodiment, the intranasal delivery device 10 can provide an intranasal delivery device that delivers a drug to the nasal cavity.

In an embodiment, the intranasal delivery device 10 can comprise a structure that contains a convex applicator.

In an embodiment, the intranasal delivery device 10 can include a convex applicator that can be inserted into the nostril.

In an embodiment, the intranasal delivery device 10 can comprise a structure that contains a convex applicator that can be inserted into the nostril to deliver a drug to the nasal cavity.

In an embodiment, the intranasal delivery device 10 can comprise a structure that contains a convex applicator and a chamber that is in communication with the convex applicator.

In an embodiment, the intranasal delivery device 10 can comprise a structure that contains a convex applicator and a chamber, wherein the chamber contains a plurality of drug doses for dosing multiple humans in sequence.

In an embodiment, the intranasal delivery device 10 can comprise a structure that contains a convex applicator and one or more chambers that are in communication with the convex applicator.

In an embodiment, the intranasal delivery device 10 can comprise a structure that contains a convex applicator and one or more chambers, wherein a plurality of chambers contains one or more doses.

In an embodiment, the intranasal delivery device 10 can comprise a structure that contains a convex applicator and one or more chambers, wherein a plurality of chambers contains the same drug or two different drugs.

In an embodiment, the intranasal delivery device 10 can comprise a structure that contains a convex applicator and one or more chambers, wherein one dose of two drugs can be administered simultaneously.

In an embodiment, the intranasal delivery device 10 can comprise a structure that contains a convex applicator, wherein the convex applicator has an antimicrobial coating.

In an embodiment, the intranasal delivery device 10 can comprise a structure that contains a convex applicator and a covering, wherein a suitable material covers the entire convex applicator except for the opening where the drug is transferred from the device to the nasal cavity.

In an embodiment, the intranasal delivery device 10 can comprise a structure that contains a convex applicator and a covering, wherein a pliable film covers the entire convex applicator except for the opening where the drug is transferred from the device to the nasal cavity.

In an embodiment, the intranasal delivery device 10 can comprise a structure that contains a convex applicator and a covering, wherein a suitable material covers the entire convex applicator except for the opening and the covering is coated with an antimicrobial.

In an embodiment, the intranasal delivery device 10 can comprise a structure that contains a convex applicator and a covering, wherein it can be replaced after dosing and a new covering is used for each patient for sequential dosing.

In an embodiment, the intranasal delivery device 10 can be utilized in a method of treating a human.

In an embodiment, the intranasal delivery device 10 can comprise a structure that contains a convex applicator and a chamber that is in communication with the convex applicator and a diffuser in communication with the chamber and convex applicator.

In an embodiment, the intranasal delivery device 10 can facilitate a method of propelling the drug to the nasal cavity.

In an embodiment, the intranasal delivery device 10 can deliver a drug to the nasal cavity, wherein the drug is a chemical drug, biological drug, chemical compound, or an antidote.

In an embodiment, the intranasal delivery device 10 can deliver a drug to the nasal cavity, wherein the drug is a liquid, gel, atomized fluid, or a lyophilized powder.

In an embodiment, the intranasal delivery device 10 can deliver a single drug or multiple drugs into one or both nostrils of a single individual or multiple individuals

In an embodiment, the intranasal delivery device 10 can comprise a structure that contains two convex applicators.

In an embodiment, the intranasal delivery device 10 can comprise a structure that contains two convex applicators that can be inserted into both nostrils of a patient simultaneously.

In an embodiment, the intranasal delivery device 10 can comprise a structure that contains two convex applicators and a chamber that is in communication with the convex applicators.

In an embodiment, the intranasal delivery device 10 can comprise a structure that contains two convex applicators and a chamber that is in communication with the convex applicators and a diffuser in communication with the chamber and convex applicators.

In an embodiment, the intranasal delivery device 10 can comprise a structure that contains two convex applicators, wherein the chamber contains a plurality of drug doses for dosing multiple humans in sequence.

In an embodiment, the intranasal delivery device 10 can comprise a structure that contains two convex applicators and one or more chambers that are in communication with the convex applicators.

In an embodiment, the intranasal delivery device 10 can comprise a structure that contains two convex applicators and a plurality of chambers, wherein a plurality of chambers can contain one or more doses.

In an embodiment, the intranasal delivery device 10 can comprise a structure that contains two convex applicators and one or more chambers, wherein a plurality of chambers that contain the same drug or two different drugs.

In an embodiment, the intranasal delivery device 10 can comprise a structure that contains two convex applicators and one or more chambers, wherein one dose of two drugs can be administered simultaneously.

In an embodiment, the intranasal delivery device 10 can comprise a structure that contains two convex applicators, wherein one or both of the convex applicators have an antimicrobial coating.

In an embodiment, the intranasal delivery device 10 can comprise two convex applicators and a covering, wherein a suitable material covers the entire convex applicators except for the opening where the drug can be transferred from the device to the nasal cavity.

In an embodiment, the intranasal delivery device 10 can comprise a structure that contains two convex applicators and a covering, wherein a pliable film covers the entire convex applicators except for the opening where the drug can be transferred from the device to the nasal cavity.

In an embodiment, the intranasal delivery device 10 can comprise a structure that contains two convex applicators and a covering, wherein a suitable material covers the entire convex applicators except for the opening and the covering can be coated with an antimicrobial.

In an embodiment, the intranasal delivery device 10 can comprise a structure that contains a convex applicator and a covering, wherein the covering can be removed and replaced after dosing, and a new covering is used for each patient for sequential dosing.

In an embodiment, the intranasal delivery device 10 can comprise a method of delivering a drug to the nasal cavity through both nostrils by a patient capable of self-administration.

In an embodiment, the intranasal delivery device 10 can comprise a method of delivering a drug to the nasal cavity through both nostrils by a first responder or caregiver.

In an embodiment, the intranasal delivery device 10 can comprise a structure that contains two convex applicators a method of delivering a drug to the nasal cavity through both nostrils simultaneously to nonhuman primate or a human.

In an embodiment an intranasal delivery device 10 can have a first and/or a second convex structure that fits into a nostril as a nasal applicator member 12. The size, strength, shape, and material of the device can be selected for safe and effective insertion into the nostril. The intranasal delivery device can be connected to and positioned above a housing 14 that can comprise materials suitable for containing an antidote, drug or compound. In an embodiment, the housing 14 can be made of materials which can withstand sterilization of the antidote, drug or compound contained within. Sterilization of the antidote, drug, compound, or housing 14 can be accomplished by heat or radiation. A safety covering 18 can be made of a suitable material to envelop the entire convex applicator except for the opening where the drug can be transferred from the device to the nasal cavity. The safety covering 18 allows for one intranasal delivery device 10 to be used to treat many individuals in sequence, while preventing the exchange of nasal bodily fluid between individuals.

In an embodiment, a single antidote, drug or compound can be delivered from a first fluid chamber 22 in housing 14.

In an embodiment, a single antidote, drug or compound can be delivered from the first fluid chamber 22 and/or the second fluid chamber 24 in housing 14.

In an embodiment, a first antidote, drug or compound can be delivered from a first fluid chamber 22, and a second antidote, drug or compound can be delivered from a second fluid chamber 24 in housing 14.

The intranasal delivery device 10 may include one or more chambers proximally connected to the device and extending distally from the device. For example, the intranasal drug delivery device may have one chamber container connected thereto. In other embodiments, the intranasal drug delivery device may have two chamber containers, three chamber containers, four chamber containers connected thereto. The one or more chamber containers may include an inlet positioned at the proximal end of the one or more chamber containers. In some embodiments, each proximal end of the one or more chamber containers can be connected to and in fluid communication with the two convex structures such that the one or more chamber containers may transport fluid within the one or more chamber containers to the one or more convex structures. The fluid exits each of the one or more convex structures to the area surrounding the convex structure for example into the nose.

In an embodiment, the intranasal delivery device 10 may have an internally segmented housing 14. Within the internally segmented housing 14 can be a plurality of dividers 20 that segment the housing 14 into a plurality of chambers, including two chambers, three chambers, four chambers, or five chambers, or more.

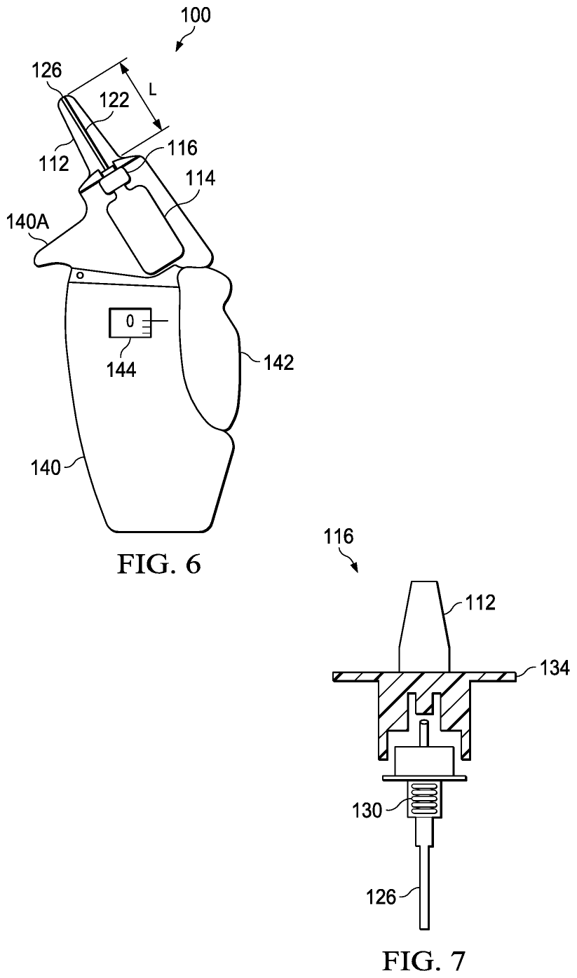

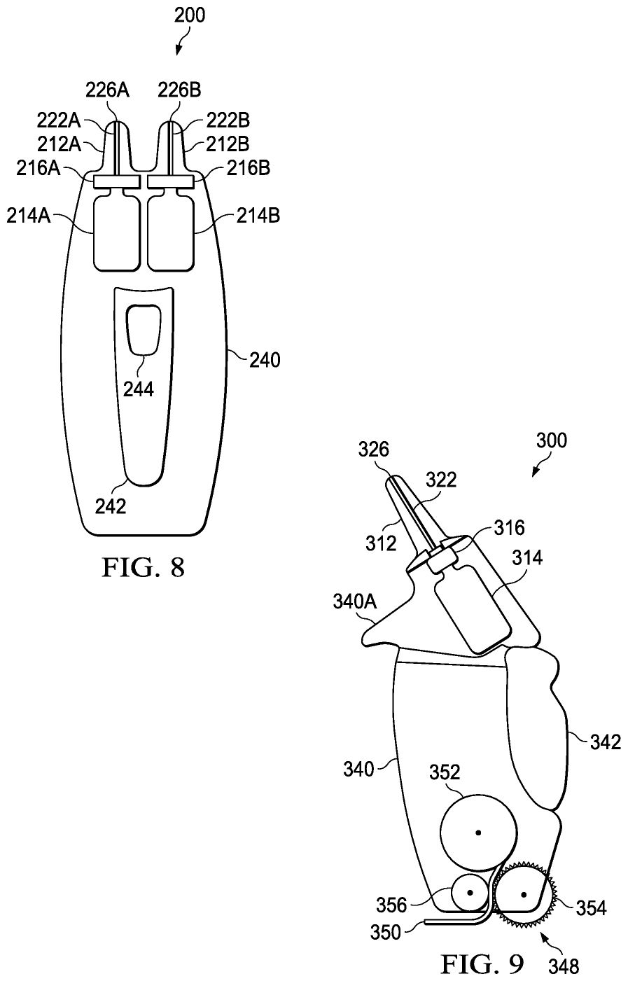

Referring now to FIG. 6, there is shown a schematic side elevation representation of an embodiment of a device 100 for intranasal delivery of an antidote. The device shown in FIG. 6 can deliver an antidote to one or both nostrils of a person in need of the antidote. That is, the device 100 can deliver an antidote contained in a housing 114 through a nasal applicator member 112. In an embodiment, a device 100 can have two nasal applicator members such as, for example, first nasal applicator member and second nasal applicator member as shown and described below with respect to in FIG. 8 as 212A and 212B. Nasal applicator member(s) 112 can be operatively associated with one or both of the housing 114 and the dispersing member 116 and can have defined therein a passageway 122 to provide fluid communication between the interior of housing 114 through the dispersing member 116, through a distal opening 126 and to the nostrils of a user. In an embodiment, housing 114 can be segmented internally by a divider (not shown) to define two discrete chambers (not shown), in each of which can be contained a fluid antidote to be released through nasal applicator member(s) 112. The fluid can be an antidote to a chemical and/or biological agent, and can be released as a mist, an atomized mist, as droplets, or combinations thereof.

The device 100 can have a handle 140 and first and second antidotes actuator 142 sized and shaped for ease of use and operationally connected to the housing 114 and/or the dispersing member 116. For example, the actuator 142 can be hingedly connected to the handle 140 and operatively associated with the housing 114 such that upon squeezing in the hand of a treatment administrator from a first actuator position to a second actuator position, the actuator 142 can be urged against housing 114, and thereupon pressed against the dispersing member 116 to subsequently dispense a treatment fluid through the nasal applicator member(s) 112. Handle 140 and actuator 142 can comprise molded plastic, for example, and can have a generally smooth sided shape for comfort and ergonomic use by a treatment administrator. This could include facilitating use while in mission oriented protective posture (MOPP) gear including personal protective equipment (PPE). In an embodiment, an upper handle portion 140A can contain housing 114 and provide for a protrusion, which can comfortably position the index finger of a user, with the remaining fingers wrapped around the lower handle portion, i.e., handle 140, and the palm of the user's hand closed on the actuator 142. In another embodiment, an upper handle portion 140A can contain housing 114 and provide for a protrusion below which can comfortably position the palm of the user's hand, with the fingers wrapped around the handle 140, and close on the actuator 142.

In addition to the features described above, a device 100 of the present disclosure can comprise a counter 144. Counter 144 can be configured to register the sequential release of a treatment fluid through nasal applicator member(s) 112. Counter 144 can be mechanical or electronic, and can be mechanically or electronically operatively connected to an actuating component, such as actuator 142. In operation, each dispensing action of the device 100 can register a count on the counter 144. The count can increase from 1, indicating a first treatment, and can increase sequentially to a number indicating a last actuation, or it can decrease from a maximum number of treatments available, to the number 1, indicating a last treatment. In an embodiment, the visual representation of the count on counter 144 can also indicate an amount of dispensing actuations left in the device 100. For example, the last five dispensing treatments can be indicated by the visual indication on the counter being the color red. Counter 144 can be attached to the device 100, such as by adhesive, or counter 144 can be integrated into the handle 140, inside handle 140, or operatively disposed on the actuator 142, upper handle portion 140A, or another portion of the device 100.

In general, nasal applicator member(s) 112 can be shaped for efficient entry into the nasal passages, i.e., the nostrils, of a person in need of an antidote. A distal end of each nasal applicator member 112 can be generally rounded and convex. The material of the nasal applicator member 112 can be flexible, pliable, and sufficiently stiff as to be properly inserted into a nasal passage, i.e., a nostril. It is contemplated that the length L of nasal applicator member(s) 112 can be from about 1 cm to about 8 cm, or from about 1 cm to about 7 cm, or from about 1 cm to about 6 cm, or from about 1 cm to about 5 cm, or from about 1 cm to about 4 cm, or from about 1 cm to about 3 cm, or from about 1 cm to about 2 cm. If two nasal applicator members are used, it is contemplated that the center-to-center (as indicated above in FIG. 3) distance between the nasal applicator members may be from about 1 cm to about 8 cm, or from about 1 cm to about 7 cm, or from about 1 cm to about 6 cm, or from about 1 cm to about 5 cm, or from about 1 cm to about 4 cm, or from about 1 cm to about 3 cm, or from about 1 cm to about 2 cm. In an embodiment, two nasal applicator members (e.g., 212A and 212B) can have a side-to-side separation distance as indicated above in FIG. 3) of from about 0.5 cm to about 5 cm, or from about 1 cm to about 4 cm, or from about 2 cm to about 3 cm.

In an embodiment, a method of using the device 100 includes dosing for single patient use with multiple doses contained within the device. As an example, the device 100 can be used to dose an individual in need of an antidote. The device can be attached to the patient with a strap, lanyard, belt clip, hook, loop fastener, and the like. Multiple doses could be delivered from the device to a patient as needed. The device 100 can comprise the counter 144 to track the number of doses administered to a patient or patients. The device could remain with the patient during transport to facilitate identification of what treatment was administered, and the number of doses administered. It is believed essential for proper healthcare that healthcare providers know what treatments patients have received so that future care can be administered appropriately.

As indicated in FIG. 7, an example dispersing member 116, can be affixed, such as by threaded connection, onto a top opening of a housing 114. The dispersing member 116 can have a pressure application member 134, which can be, for example, an extension of the dispersing member and upon which the fingers of a user can squeeze against forces exerted by spring (not shown), or as shown in FIG. 1, interiorly disposed for pressure against an interior portion of the device 100. When urged against the spring force of spring, a dose of an antidote from fluid chamber, such as housing 114 can be communicated through and released through the nasal applicator member(s) 112 to the nostril(s) of a patient. The dispersing member 116 can include a spring-loaded valve having a first position in which fluid communication between an interior chamber and a nasal applicator member is blocked, and a second position in which fluid communication between an interior chamber and a nasal applicator member is open. Likewise, for embodiments having two-nasal applicator members, the dispersing member can include a spring-loaded valve having a first position in which fluid communication between a first and second interior chamber and first and second nasal applicator members, respectively, are blocked, and a second position in which fluid communication between a first and second interior chamber and the first and second nasal applicator members, respectively, are open. The valve can also be pressure activated, and could be open, for example, by the action of squeezing the housing 114, which can be a flexible, squeezable container, to provide internal positive pressure that can open the valve and find fluid passageway to the nasal applicator member(s).

In general, the dispersing member 116 can have and utilize any of known pumps, pistons, springs, stored energy, and can operate electrically, magnetically, pneumatically or hydraulically, or with other forms of energy, as is known for nasal applicator devices.

Referring now to FIG. 8, there is shown a schematic front elevation representation of an embodiment of a device 200 for intranasal delivery of an antidote. As shown in FIG. 8, the device 200 can be as described above, but can optionally have two nasal applicator members, a first nasal applicator member 212A and a second nasal applicator member 212B, such that upon use, a fluid antidote from one housing, or two discrete housings, e.g., a first fluid chamber 214A and a second fluid chamber 214B, can be dispensed through and out of one or both of the nasal applicator members, and ultimately into one or both nostrils. First nasal applicator member 212A can have defined therein a passageway, such as first passageway 222A, to provide fluid communication between the first fluid chamber 214A through a first dispersing member 216A, through a first distal opening 226A to the nostril of a user. Likewise, second nasal applicator member 212B can have defined therein a passageway, such as second passageway 222B, to provide fluid communication between the second fluid chamber 214B through a second dispersing member 216B, through a second distal opening 226B to the nostril of a user. Each of the first fluid chamber 214A and second fluid chamber 214B can be distinct containers, or a single container with the housings being defined by a separating divider of a housing (not shown). As shown, a counter 244 can be disposed on actuator 242. The actuator 242 can be a pressure activating mechanism that, when pressed, pushed, or otherwise urged inwardly with respect to the handle 240, can cause operation of a first valve in the first dispersing member 216A and/or the second valve in the second dispersing member 216B, to disperse of an antidote from one or both of the first fluid chamber 214A and the second fluid chamber 214B.

Referring now to FIG. 9, there is shown a schematic side elevation representation of an embodiment of a device 300 for intranasal delivery of an antidote. As shown in FIG. 9, the device 300 can be as described above, including at least one nasal applicator member 312, such that upon use, a fluid antidote from a housing 314 can be dispersed via dispersing member 316 through the nasal applicator member 312, and ultimately into a nostril. The nasal applicator member 312 can have defined therein a passageway 322 to provide fluid communication between the interior of housing 314 through a dispersing member 316, through a distal opening 326 and to the nostrils of a user. An actuator 342 can be a pressure activated mechanism that, when pressed, pushed, or otherwise urged inwardly with respect to the handle 340, can cause dispersing of an antidote from the housing 314. In an embodiment, an upper handle portion 340A can contain housing 314 and provide for a protrusion, which can comfortably position the index finger of a user, with the remaining fingers wrapped around the lower handle portion, i.e., handle 340, and the palm of the user's hand closed on the actuator 342.

As shown in FIG. 9, the device 300 can be as described above, but can optionally have a label dispenser 348 disposed in or on the device 300, for example inside the handle 340. The label dispenser 348 can have a label spool 352 on which can be spooled a supply of a label strip 350, which can be printed on upon dispensing, written on upon dispensing, or pre-printed with patient identifying indicia and/or treatment information, such as time, date, dose, and treatment type. A thumbwheel 354 can be rotated by a caregiver to force dispensing of the label strip 350 through a nip defined by contact of the thumbwheel 354 with a backing wheel 356. Once dispensed, a portion of the label strip 350 can be removed and affixed on a patient, such as on a patient's person or clothing. The portion of the label strip 350 affixed onto a patient can, for example, in addition to identifying the patient (if such information is known), identify the treatment administered to the patient. The label spool 352 can be located anywhere in or on the device 300, but can be disposed within the handle 340 opposite the nasal applicator member 312, near the bottom of the handle 340, such that the portion of the label strip 350 may exit the bottom of the handle 340, as shown in FIG. 4. The portion of the label strip 350 can be manually applied by pressure or by other mechanisms such as a roller that rotates and dispenses an adhesive label when pressed against a surface such as the body of a patient, for example.

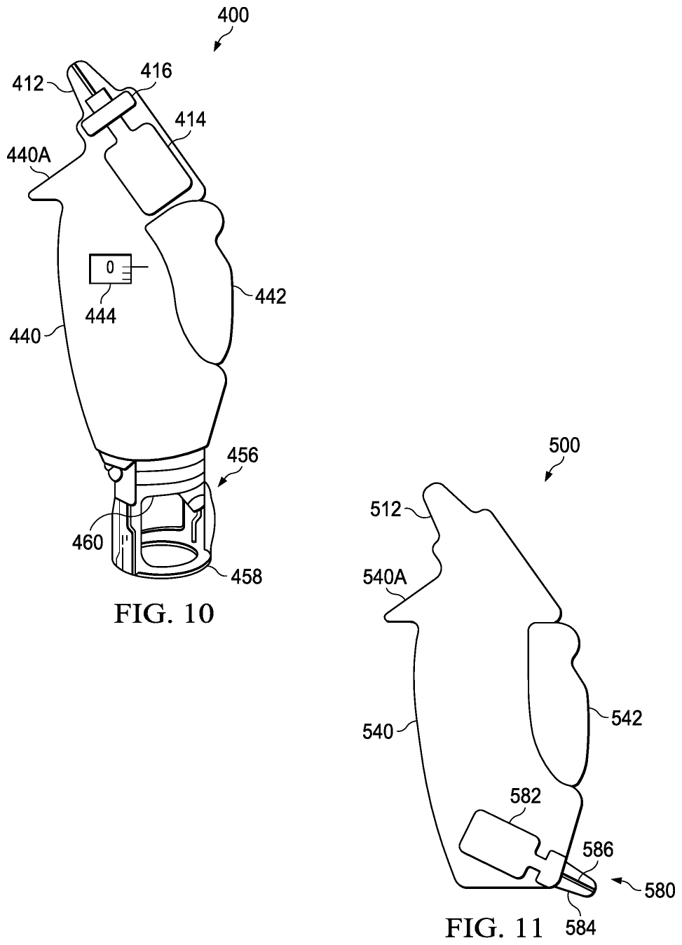

As shown in FIG. 10, an example device 400 can be as described above, including having a handle 440 and actuator 442 sized and shaped for ease of use. As described above, the actuator 442 can be hingedly connected to the handle 440 and operatively associated with a housing 414 as described above, such that upon squeezing in the hand of a treatment administrator, the actuator 442 can be urged against the housing 414, and thereupon pressed against the actuator as described above, to subsequently dispense a treatment fluid through the nasal applicator member 412. Handle 440 and actuator 442 can comprise molded plastic, for example, and can have a generally smooth sided shape for comfort and ergonomic use by a treatment administrator. In an embodiment, an upper handle portion can provide for a protrusion 440A, which can comfortably position the index finger of a user, with the remaining fingers wrapped around the lower handle portion, i.e., handle 440, and the palm of the user's hand closed on the actuator 442. In another embodiment, an upper handle portion can contain housing 414 and provide for a protrusion below which can comfortably position the palm of the user's hand, with the fingers wrapped around the handle 440, and close on the actuator 442.

In addition to the features described above, a device 400 of the present disclosure can comprise marking fluid deposition member, such as an ink stamping member 456. Ink stamping member 456 facilitates the stamping of a marking fluid such as a dye or ink, onto a fluid deposition surface, such as the skin or clothing of a patient, such as with patient identifying indicia and/or treatment information, such as time, date, dose, route of administration, and treatment type. The ink stamping member 456 can include a surface contacting member 458, which upon being urged against the fluid deposition surface, can be moved, for example against a spring force, toward the handle 440, thereby activating an ink stamp 460, which can rotate from a closed, ink preserving state, to an open, fluid deposition surface-facing printing state. The ink stamp can contact the fluid deposition surface, thereby depositing the patient identifying indicia and/or treatment information. In another embodiment, an upper handle portion can contain housing 414 and provide for a protrusion below which can comfortably position the palm of the user's hand, with the fingers wrapped around the handle 440, and close on the actuator 442.

As shown in FIG. 11, an example device 500 can be as described above, including having a handle 540 and actuator 542 sized and shaped for ease of use. As described above, the actuator 542 can be hingedly connected to the handle 540 and operatively associated with a housing (not shown) as described above, such that upon squeezing in the hand of a treatment administrator, the actuator 542 can be urged against the housing, and thereupon pressed against the actuator as described above, to subsequently dispense a treatment fluid through the nasal applicator member 512. Nasal applicator member 512 can have defined therein a passageway (not shown, but as described above) to provide fluid communication between the interior of housing through a dispersing member, through a distal opening, and to the nostrils of a user. Handle 540 and actuator 542 can comprise molded plastic, for example, and can have a generally smooth sided shape for comfort and ergonomic use by a treatment administrator. In an embodiment, an upper handle portion can provide for a protrusion 540A, which can comfortably position the index finger of a user, with the remaining fingers wrapped around the lower handle portion, i.e., handle 540, and the palm of the user's hand closed on the actuator 542.

In addition to the features described above, a device 500 of the present disclosure can comprise a marking fluid spray member, such as an ink spray member 580. Ink spray member 580, can facilitate the dispersing, such as a spray, of an ink, dye, or other indicia-forming fluid from a fluid container 582 through a fluid spray nozzle 584. In operation, the ink spray member 580 can be located at the bottom of the handle 540, as shown in FIG. 11. After administration of a treatment dose of an antidote, for example as described above, the caregiver can, for example, activate the ink spray member 580 to emit an indicia-forming fluid through an internal passageway 586 to a patient, such as onto a patient's skin, clothing, or paper associated with the patient. The fluid container 582 can include an ink, including a durable ink, which can facilitate the marking of a patient with patient identifying indicia and/or treatment information, such as time, date, dose, route of administration, and treatment type. The ink spray member 580 can, for example, provide a spray of dye on the patient in sequence or after treatment with the device to identify the treatment administered for first responders to identify patients that have received treatment and for those healthcare providers that may provide care for the patient after transport to a healthcare facility. The dye can be located within the fluid container 582 and a spraying mechanism, such as squeezing or otherwise deforming the fluid spray nozzle 584, can activate the delivery of the marking spray, for example, to the skin of an individual.

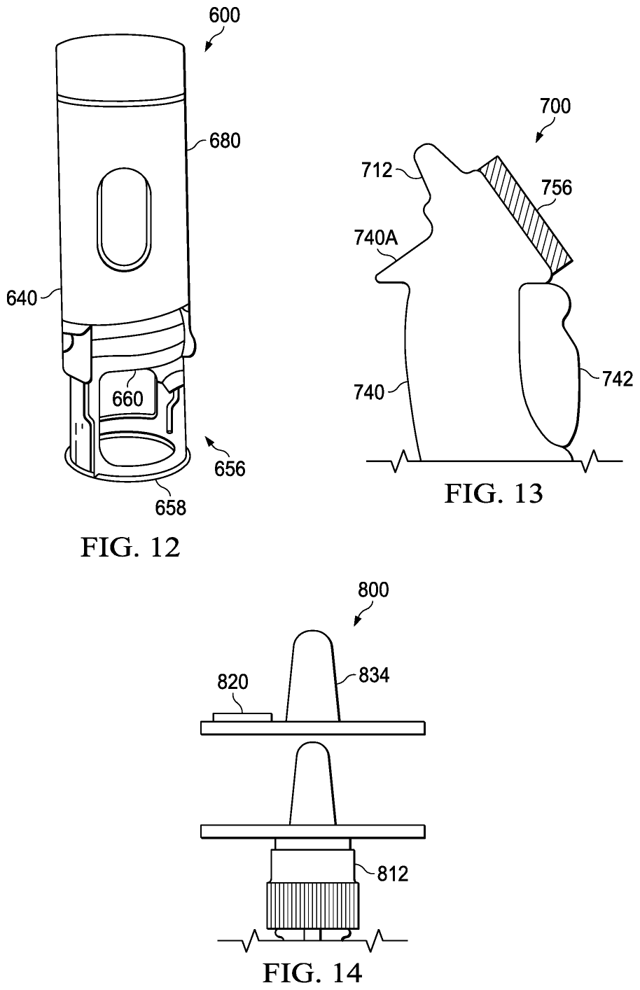

As shown in FIG. 12, in an embodiment, a device 600 can be an autoinjector 680 rather than an intranasal device. The autoinjector 680 can be used as is known in the art to inject an antidote, for example, into a patient in need of the antidote. However, the autoinjector 680 can have any of the features described above with respect to intranasal devices, including a label dispenser, a stamping member, a spray member for facilitating the spray of an ink, dye, or other indicia-forming fluid, and/or, ink stamping member 656, as shown in FIGS. 9-11. Ink stamping member 656 facilitates the stamping of a marking fluid such as a dye or ink, onto a fluid deposition surface, such as the skin or clothing of a patient, such as with patient identifying indicia and/or treatment information, such as time, date, dose, route of administration, and treatment type. The ink stamping member 656 can include a surface contacting member 658, which upon being urged against the fluid deposition surface, can be moved, for example against a spring force, toward a handle 640, thereby activating an ink stamp 660, which can rotate from a closed, ink preserving state, to an open, fluid deposition surface-facing printing state. The ink stamp 660 can contact the fluid deposition surface, thereby depositing the patient identifying indicia and/or treatment information onto the fluid deposition surface.

Referring now to FIG. 13, there is shown schematic side elevation representation of an embodiment of a device 700 for intranasal delivery of an antidote. As shown in FIG. 13, the device 700 can be as described above, including at least one nasal applicator member 712, such that upon use, a fluid antidote from a housing can be dispersed via a dispersing member through the nasal applicator member 712, and ultimately into a nostril. An actuator 742 can be a pressure activated mechanism that, when pressed, pushed, or otherwise urged inwardly with respect to the handle 740, can cause dispersing of an antidote from the housing. In an embodiment, an upper handle portion 740A can contain the housing and provide for a protrusion, which can comfortably position the index finger of a user, with the remaining fingers wrapped around the lower handle portion, i.e., handle 740, and the palm of the user's hand closed on the actuator 742.

As shown in FIG. 13, the device 700 can be as described above, but can optionally have an ink containing member 756 disposed in or on the device 700, for example on the handle 740. Ink containing member 756 can be an ink stamp pad. Ink containing member 756 can facilitate the deposition of a marking fluid such as a dye or ink. Ink deposition can be used to mark a patient having received an intranasal dose with identifying indicia and/or treatment information, such as time, date, dose, route of administration, and treatment type. The ink containing member 756 can be an ink stamp used to stamp ink onto a surface such as a patient's skin, such as the nostril, upper lip, lips, or chin as well as on patient's clothing or gear during or after use of the device 700. An ink stamp can contact the fluid deposition surface, thereby depositing the patient identifying indicia and/or treatment information.

As shown in FIG. 14, a safety device 800 can be utilized on any of the devices described herein. For example, a removable safety covering 834 can be provided to cover a nasal applicator member 812 before use. The safety covering can be, for example, a pliable film. In an embodiment, a first safety covering can cover the all or part of nasal applicator member 812 except for an opening where the antidote can be transferred from the device to the nasal cavity and which can be removed and replaced with a second safety covering (not shown) before dosing another individual. In this manner, the exchange of transmittable viruses and bacteria between individuals can be minimized or eliminated. The removable safety covering 834 can include a marking fluid 820 such as a dye or ink that when inserted into the nostril or nostrils can transfer to the patient to leave an identifiable indicia-forming fluid onto a patient's skin, such as the nostril, upper lip, lips, or chin. The marking fluid 820 can include an ink, including a durable ink, which can facilitate the marking of a patient with patient identifying indicia and/or treatment information, such as time, date, dose, route of administration, and treatment type. The removable safety covering 834, for example, may leave a transfer dye on the patient to identify the treatment administered for those healthcare providers that may provide care for the patient after transport to healthcare facility.

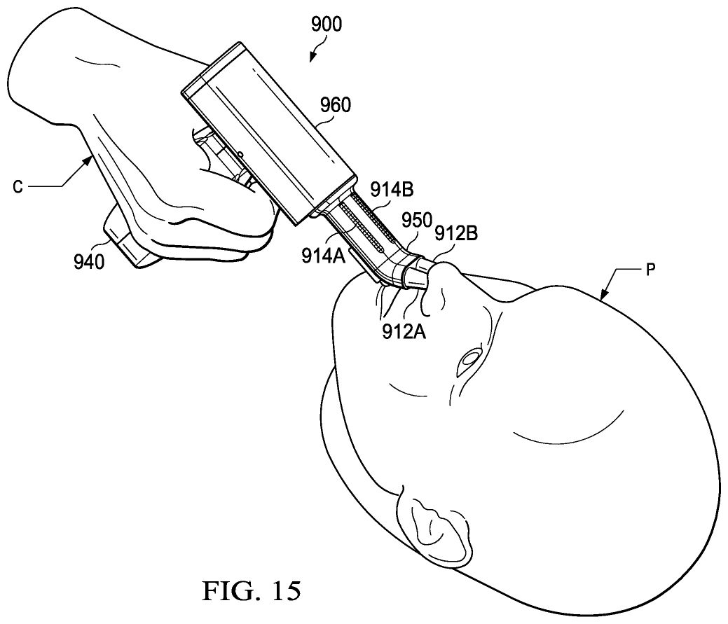

Referring now to FIGS. 15-19, there is described an embodiment of a device 900 for intranasal delivery of an antidote to a patient. The device 900, described in more detail below, features an ergonomic design and reliable sequential multi-patient dosing. The multi-use design facilitates stockpile storage of large quantities of antidotes and can be used to meet the specific countermeasure treatments for civilian and military first responders. The device 900 can include the ability to deliver doses to multiple patients in sequence, delivery of one or more drugs into both nostrils simultaneously, and the ability to deliver wide range of volumes of antidotes, including the ability to deliver an antidote into both nostrils simultaneously. The device has two nasal applicators that each have an internal fluid passage from a drug chamber. Two separate drug chambers can each contain the same drug or different drugs. The drug, or other fluid, can be an antidote to a chemical agent and can be released as a mist or atomized mist. The mechanism for dosing is a one-way syringe movement in which one activation of the “pistol-grip” style handle by squeezing can provide a defined dose through a direct ratcheting system. This is accomplished by advancing internally disposed syringes a predefined distance that is consistent with the desired dose. The desired volume of the dose can be achieved by two mechanisms or a combination of the two which includes varying the size of the syringe (and drug chamber) and/or by adjusting the movement distance of the syringe associated with the one-way syringe advancing system. These design features allow the device 900 to deliver the desired dose of a drug and allow for customization of the desired dose. If two separate antidotes are being administered, two separate dose volumes can be delivered simultaneously with one trigger dose.

Another component of the device 900, more fully described below, is a marking member that can mark the patient with a dye or ink when the device 900 is used to administer a drug into the nostrils of a patent. The marking can be used to identify a patient that received treatment by care givers, including first responders. Another feature of the device 900 is a viewing window which can be used as a visual indicator of various features, including allowing a first responder to ensure dosing is occurring and to view the number of doses remaining in the unit. In an embodiment, the device trigger will not operate when the device is empty so the first responder can identify when the device is empty.

Referring again to FIG. 15, there is shown a patient P in need of treatment being administered a drug dose by a care giver C, which can be a first responder. The device 900 is generally shaped like a pistol, with a main body 960 and a handle 940 that can be gripped by the care giver for actuation by squeezing. The drug(s) to be administered can be contained in a first fluid chamber 914A to be administered through a first nasal applicator member 912A, and a second fluid chamber 914B to be administered through a second nasal applicator member 912B. The nasal applicator members can be housed in a nasal applicator housing 950 that extends from the main body 960 at a proximal portion and can have a shape, such as the curvature indicated in FIGS. 15-19, such that a distal portion connected to the nasal applicator members is angled for safe, convenient, and ergonomically beneficial insertion into the nostrils of a patient P. Each of the components of the device 900 can comprise any of suitably durable, safe materials, including molded plastic, for example, and can have a generally smooth sided shape for comfort and ergonomic use by a treatment administrator. This could include facilitating use while in mission oriented protective posture (MOPP) gear including personal protective equipment (PPE).

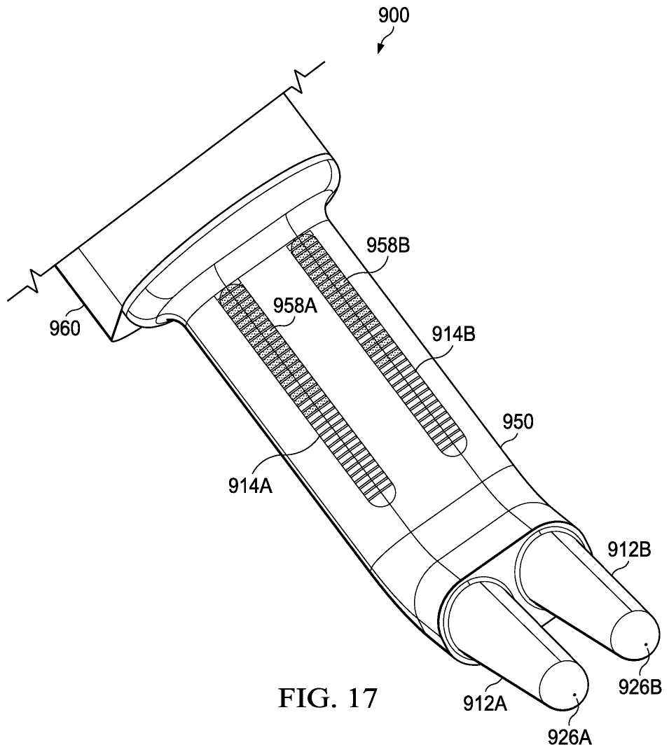

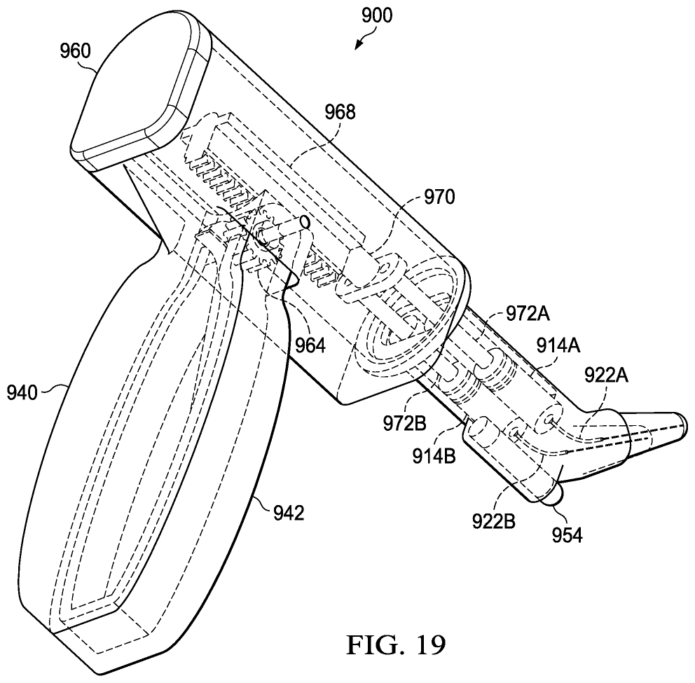

The device 900 can deliver an antidote contained in the main body 960 and/or the nasal applicator housing 950, such as a first drug in the first fluid chamber 914A and/or a second drug (that can be the same as the first drug) in the second fluid chamber 914B. The drugs can be delivered, respectively, to the nostrils of a patient in need of treatment, from the first nasal applicator member 912A and the second nasal applicator member 912B. As shown in FIG. 19, the device 900 can have defined therein a first passageway 922A and a second passageway 922B extending from the first fluid chamber 914A and the second fluid chamber 914B, respectively, to provide fluid communication between the interior of the respective fluid chambers to, respectively, the first distal opening 926A and/or second distal opening 926B (as shown in FIG. 17), and to the nostrils of a user. The drug chambers can contain any fluid, including a fluid that can be an antidote to a chemical and/or biological agent, and can be released as a mist, an atomized mist, as droplets, or combinations thereof.

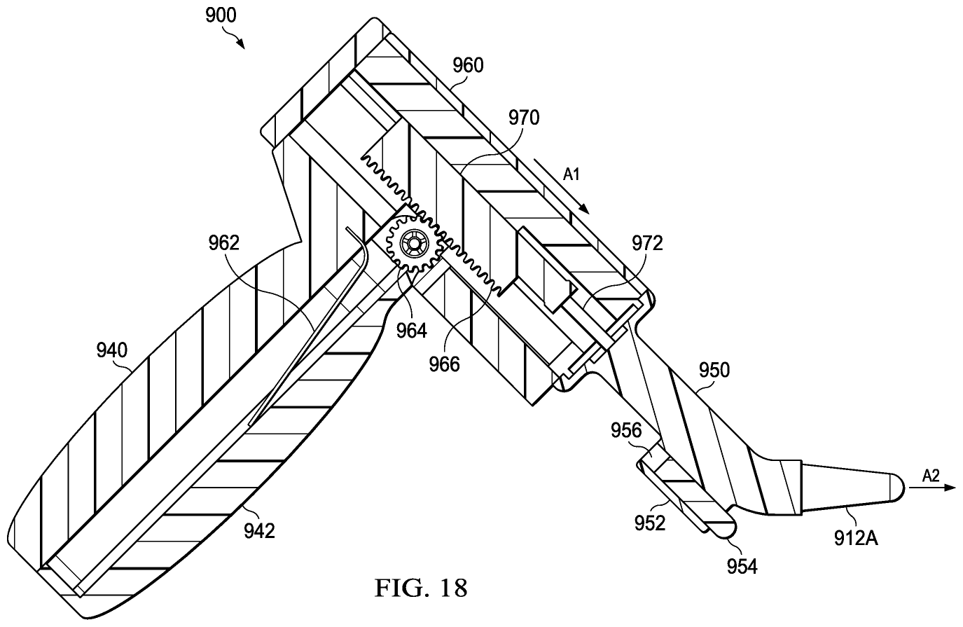

Referring primarily to the cut-away schematic view of FIG. 18 and the perspective schematic view of FIG. 19, the device 900 can have a handle 940 and actuator 942 sized and shaped generally at an angle to the main body 960, including generally orthogonally to the main body 960 as indicated in FIGS. 15-19, for ease of use in the grip of a treatment administrator, such as a first responder. For example, the actuator 942 can be moveably connected to the handle 940 and spring-biased in an open state by a spring, such as spring 962. That is, the actuator can be connected to the handle and elastically biased away from the handle, such as by the spring 962. The actuator 942 can be operatively associated such as by being hingedly connected, such that upon squeezing in the hand of a treatment administrator, the actuator 942 can be urged toward the handle 940 against the spring force of spring 962, while simultaneously activating movement of a syringe depressor 970. A portion of the actuator, for example, can be connected by a mechanical linkage to the syringe depressor 970, including, in an embodiment, with a mechanical advantage such that a slight movement of the actuator can be leveraged into a relatively large linear motion of the syringe depressor 970. In an embodiment, squeezing the actuator toward the handle simultaneously rotates a pinion gear 964 that engages a rack gear 966 that is attached to, or is a part of, the syringe depressor 970. Upon squeezing the actuator 942 toward the handle 940, the pinion gear rotates (clockwise, as depicted in FIG. 18), thereby causing the syringe depressor 970 to move in the direction of the arrow A1 in FIG. 18, causing treatment fluid to be dispensed through the nasal applicator member(s) 912 in the direction of arrow A2.

A marking device can include an ink containing member in the form of a housing 952 having an interior compartment 956 that contains a marker 954 that can mark the patient with a dye or ink when the device 900 is used to administer a drug into the nostrils of a patent. The marker 954 can be configured to be disposed on the device 900, such as on the nasal applicator housing 950, and placed at a location such that upon insertion of the nasal applicator member(s) 912 into the nostrils of a patient, the marker 954 is urged against the patient, such as on the skin, including the skin under the lower lip of the patient, as would be the case in the configuration shown in FIGS. 15-19. Thus, the marker 954 can contain, dispense, or otherwise impart an ink or a dye to the patient upon insertion of a nasal applicator member 912 into the nostril of the patient. In an embodiment, an ink or a dye can be dispensed from the marker 954 when the marker is deformed, such as by being pressed against the skin of the user.

In general, for each of the intranasal delivery devices disclosed herein, another device and method for marking a dosed patient can include providing an ink or dye directly onto the patient-contacting surface of the removable safety covering, as disclosed in FIG. 14, with removable safety covering 834 including a marking fluid 820 such as a dye or ink that when inserted into the nostril or nostrils can transfer to the patient to leave an identifiable indicia-forming fluid onto a patient's skin, such as the nostril, upper lip, lips, or chin. The dye or ink (or other marker medium) can also be antimicrobial.

As can be understood from the description herein, therefore, the device 900 can be handled in an ergonomic fashion by a first responder to provide a dose of a fluid treatment to a patient by the action of squeezing the handle 940, which can be a pistol-grip style handle, that drives the syringe depressor 970 to force a syringe 972 to displace a fluid in one or both of the first fluid chamber 914A and the second fluid chamber 914B. Thus, there can be two syringes, a first syringe 972A that displaces a first fluid in the first fluid chamber 914A and a second syringe 972B that displaces a second fluid in the second fluid chamber 914B, as best seen in FIG. 19. The syringe depressor 970 can be operatively connected to both the first syringe 972A and the second syringe 972B, and can have sliding bearing surfaces 968 that serve to position and guide the linear movement of the syringe depressor 970. In an embodiment, the movement of the syringe depressor 970 is achieved by the rack and pinion gearing described herein, and can be considered a “ratcheting” mechanism, in that the syringe depressor 970 can be urged in one direction only, i.e., the direction of the arrow A1 in FIG. 18. Thus, as squeezing pressure on the actuator 942 is relaxed and the spring 962 urges the actuator 942 away from the handle 940, the syringe depressor 970 does not advance in any direction, but stays in position, ready to be forced with the next squeeze of the handle to force another dose of the fluid from the first fluid chamber 914A and/or the second fluid chamber 914B. Other mechanisms for one-way movement of the syringe depressor 970 can be implemented to deliver the “ratcheting” single-does delivery benefit to the device 900.

The device can have on a portion thereof, such as on the nasal applicator housing 950 at least one visual indicator to visually indicate the presence of, the dispensing of, and/or the amount of fluid in one or both of the first fluid chamber 914A and/or the second fluid chamber 914B. As indicated in FIG. 17, the visual indicator can be a transparent portion, such as a window, through which a care giver can visually see an indication of the fluid. A first window 958A can provide a visual indication of the fluid in first fluid chamber 914A, and a second window 958B can provide a visual indication of the fluid in second fluid chamber 914B.

In addition to the features described above, a device 900 of the present disclosure can comprise any of the features disclosed herein, including a counter that can be configured to register the sequential release of a treatment fluid through nasal applicator member(s) 912. The counter can be mechanical or electronic, and can be mechanically or electronically operatively connected to an actuating component, such as the actuator 942. In operation, each dispensing action of the device 900 can register a count on the counter. The count can increase from 1, indicating a first treatment, and can increase sequentially to a number indicating a last actuation, or it can decrease from a maximum number of treatments available, to the number 1, indicating a last treatment. In an embodiment, the visual representation of the count on the counter can also indicate an amount of dispensing actuations left in the device 900. For example, the last five dispensing treatments can be indicated by the visual indication on the counter being the color red. The counter can be attached to the device 900, such as by adhesive, or the counter can be integrated into the handle 940, inside handle 940, or operatively disposed on the actuator 942.

In general, as described above for other example embodiments, nasal applicator member(s) 912 can be shaped for efficient entry into the nasal passages, i.e., the nostrils, of a person in need of an antidote. A distal end of each nasal applicator member 912 can be generally rounded and convex. The material of the nasal applicator member 912 can be flexible, pliable, and sufficiently stiff as to be properly inserted into a nasal passage, i.e., a nostril. Further, as discussed above, the device 900 can incorporate a removable safety covering which can provide protection for multiple users of the device in sequence. The safety covering can be made of a pliable film and can cover the nasal applicator member(s) except for distal openings 926 where the fluid, such as an antidote, is transferred from the device to the nasal cavity of a patient. In addition to the safety covering, the entire nasal applicator member(s) could be removed and replaced between patient dosing. The safety covering or nasal applicator can be removed and replaced before dosing each patient. In an embodiment, removal of the safety covering or the nasal applicator, or other portion of the intranasal delivery device can be automated, such as having a release mechanism trigger removal when the actuator is urged away by spring action from the handle. By removing patient-contacting and patient fluid-contacting portions, the exchange of transmittable viruses and bacteria between individuals can be minimized or eliminated.

As can be understood, therefore, the device 900 can administer an intranasal dose to both nostrils of a patient simultaneously. It is believed that a limited number of drugs meet the criteria for intranasal administration since drugs must be concentrated so that a dose can be administered in a relatively small total volume. Intranasal drugs are commonly administered through one nostril or through both nostrils separately for larger doses of a single drug. For drugs absorbed through the nasal mucosal, the larger the mucosal surface area, the more drug that can be absorbed. In an embodiment, the intranasal dose of a single drug can be divided in half, and each nostril can receive half the dose to essentially double the absorptive surface area. Thus, the device 900 overcomes a limitation of current intranasal delivery devices with the ability to administer a relatively large single dose of one drug in both nostrils simultaneously. Moreover, multiple antidotes can be administered either at the same time or in close temporal sequence in response as required, such as after a chemical attack. For example, pralidoxime and atropine must be administered together for the treatment of poisoning by organophosphorus nerve agents. This is typically achieved by two auto-injectors or a dual auto-injector that can administer both doses simultaneously. The device 900 can administer medications in both nostrils and provides a method and system for dosing two different antidotes simultaneously.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art. Rather it is hereby intended the scope of the invention to be defined by the claims appended hereto.

Representative embodiments of the present disclosure described above can be described as follows:
A. An intranasal delivery device, comprising:
   a housing defining a first fluid chamber, a second fluid chamber separated by a divider from the first fluid chamber, and an opening;
   a dispersing member attached to the opening on the housing, the dispersing member having a first tube extending into the first fluid chamber and a second tube extending into the second fluid chamber;
   a valve in the dispersing member, the valve having a first closed state and a second open state;
   a first fluid in the first fluid chamber and a second fluid in the second fluid chamber, the first fluid being a first antidote and the second fluid being a second antidote;
   a nasal applicator member attached to the dispersing member; and
   wherein in the first closed state the valve blocks fluid communication between the first fluid chamber and the nasal applicator member, and in the second open state the valve facilitates fluid communication between the first fluid chamber and the nasal applicator member is open.
B. The intranasal delivery device of paragraph A, wherein the nasal applicator member is flexible, having a generally rounded distal end.
C. The intranasal delivery device of paragraph A, wherein one of the first antidote and the second antidote is an antidote for a chemical agent.
D. The intranasal delivery device of paragraph A, wherein one of the first antidote and the second antidote is an antidote for a biological agent.
E. The intranasal delivery device of paragraph A, wherein the dispersing member comprises a pressure application member.
F. The intranasal delivery device of paragraph A, wherein the valve is spring biased in the first closed state.
G. The intranasal delivery device of paragraph A, wherein in the second open state the valve facilitates fluid communication of a single dose of a first antidote from the first fluid chamber.
H. The intranasal delivery device of paragraph A, wherein in the second open state the valve facilitates fluid communication of a single dose of a second antidote from the second fluid chamber.
I. The intranasal delivery device of paragraph A, further comprising a pliable film covering the nasal applicator member, the pliable film defining an opening through which fluid dispensed from the nasal applicator member can pass.
J. The intranasal delivery device of paragraph A, further comprising a pliable film covering the nasal applicator member, the pliable film defining an opening through which fluid dispensed from the nasal applicator member can pass, the pliable film further comprising an antimicrobial coating.
K. The intranasal delivery device of paragraph A, further comprising a counter configured to register a sequential release of one of the first fluid and the second fluid.
L. The intranasal delivery device of paragraph A, further comprising a label dispenser operational associated with the intranasal delivery device.
M. The intranasal delivery device of paragraph A, further comprising a marking fluid deposition member.
N. The intranasal delivery device of paragraph A, further comprising a marking fluid deposition member, the marking fluid deposition member being an ink stamping member.
O. The intranasal delivery device of paragraph A, further comprising a marking fluid deposition member.
P. The intranasal delivery device of paragraph A, further comprising a marking fluid spray member, the marking fluid spray member being an ink spray member.
Q. An intranasal delivery device, comprising:
   a handle;
   a first housing disposed in the handle, the first housing defining a first fluid chamber and an opening;
   a first nasal applicator member joined to the first housing;
   a dispersing member attached to the opening, the dispersing member having a first tube extending into the first fluid chamber;
   a valve in the dispersing member, the valve having a first closed state and a second open state, wherein in the first closed state the valve blocks fluid communication between the first fluid chamber and the first nasal applicator member, and in the second open state the valve facilitates fluid communication between the first fluid chamber and the first nasal applicator member;
   a first fluid in the first fluid chamber, the first fluid being a first antidote; and
   an actuator movably connected to the handle, the actuator being movable from a first actuator position to a second actuator position to move the valve to the second open state.
R. The intranasal delivery device of paragraph Q, wherein the first nasal applicator member is flexible, having a generally rounded distal end.
S. The intranasal delivery device of paragraph Q, wherein the first antidote is an antidote for a chemical agent.
T. The intranasal delivery device of paragraph Q, wherein the first antidote is an antidote for a biological agent.
U. The intranasal delivery device of paragraph Q, wherein the valve is spring biased in the first closed state.
V. The intranasal delivery device of paragraph Q, wherein in the second open state the valve facilitates fluid communication of a single dose of a first antidote from the first fluid chamber.
W. The intranasal delivery device of paragraph Q, further comprising a pliable film covering the first nasal applicator member, the pliable film defining an opening through which fluid dispensed from the first nasal applicator member can pass.
X. The intranasal delivery device of paragraph Q, further comprising a pliable film covering the first nasal applicator member, the pliable film defining an opening through which fluid dispensed from the first nasal applicator member can pass, the pliable film further comprising an antimicrobial coating.
Y. The intranasal delivery device of paragraph Q, further comprising a counter configured to register a sequential release of the first fluid.
Z. The intranasal delivery device of paragraph Q, further comprising a label dispenser operational associated with the intranasal delivery device.
AA. The intranasal delivery device of paragraph Q, further comprising a marking fluid deposition member.
BB. The intranasal delivery device of paragraph Q, further comprising a marking fluid deposition member, the marking fluid deposition member being an ink stamping member.
CC. The intranasal delivery device of paragraph Q, further comprising a marking fluid deposition member.
DD. The intranasal delivery device of paragraph Q, further comprising a marking fluid spray member, the marking fluid spray member being an ink spray member.
EE. A method for delivering an antidote to a plurality of patients in need of an antidote, the method comprising the steps of:
   providing an intranasal delivery device, comprising:
      a housing defining a first fluid chamber containing a first antidote, a second fluid chamber containing a second antidote, and an opening;
      a dispersing member attached to the opening on the housing;
      a first nasal applicator member positioned to receive the first antidote from the dispersing member;
      a second nasal applicator member positioned to receive the second antidote from the dispersing member;
      a first cover on at least one of the first nasal applicator member and second nasal applicator member; and
      wherein the dispersing member comprises a valve, the valve having a first closed state and a second open state, wherein in the first closed state the valve blocks fluid communication between the first fluid chamber and the second fluid chamber and the first nasal applicator member and second nasal applicator member, respectively, and in the second open state the valve facilitates fluid communication between first fluid chamber and the second fluid chamber to the first nasal applicator member and the second nasal applicator member;
   inserting the first nasal applicator member and second nasal applicator member into nostrils of a first patient;
   urging the valve from the first closed state to the second open state to dose the first patient with both the first antidote and the second antidote;
   removing the first cover;
   providing a second cover;
   placing the second cover over at least one of the first nasal applicator member and second nasal applicator;
   inserting first nasal applicator member and second nasal applicator into nostrils of a second patient; and
   urging the valve from the first closed state to the second open state to dose the second patient with both the first antidote and the second antidote.
FF. The method of paragraph EE, wherein the first antidote and the second antidote comprise the same antidote.
GG. The method of paragraph EE, wherein the first antidote is different from the second antidote.

What is claimed is:

1. An intranasal delivery device, comprising:
a housing defining a first fluid chamber, a second fluid chamber separated from the first fluid chamber, and an opening;
a dispersing member attached to the opening on the housing, the dispersing member having a first tube extending into the first fluid chamber and a second tube extending into the second fluid chamber;
a valve in the dispersing member, the valve having a first closed state and a second open state;
a first fluid in the first fluid chamber and a second fluid in the second fluid chamber, the first fluid being a first antidote and the second fluid being a second antidote;
a first nasal applicator member attached to the dispersing member and in fluid communication with the first tube;
a second nasal applicator member attached to the dispersing member and in fluid communication with the second tube; and
wherein in the first closed state the valve blocks fluid communication between the first fluid chamber and the first nasal applicator member and the second fluid chamber and the second nasal applicator member, and in the second open state the valve facilitates fluid communication between the first fluid chamber and the first nasal applicator member is open.

2. The intranasal delivery device of claim 1, wherein the dispersing member comprises a pressure application member.

3. The intranasal delivery device of claim 1, wherein the valve is spring biased in the first closed state.

4. The intranasal delivery device of claim 1, wherein in the second open state the valve facilitates fluid communication of a single dose of a first antidote from the first fluid chamber.

5. The intranasal delivery device of claim 1, wherein in the second open state the valve facilitates fluid communication of a single dose of a second antidote from the second fluid chamber.

6. The intranasal delivery device of claim 1, further comprising a marking fluid deposition member.

7. An intranasal delivery device, comprising:
a handle;
a first housing disposed in the handle, the first housing defining a first fluid chamber and a first opening;
a first nasal applicator member joined to the handle;
a second housing disposed in the handle adjacent the first housing, the second housing defining a second fluid chamber and a second opening;
a second nasal applicator member joined to the handle;
a first dispersing member attached to the first opening, the first dispersing member having a first tube extending into the first fluid chamber;
a second dispersing member attached to the second opening, the second dispersing member having a second tube extending into the second fluid chamber;
a first valve in the first dispersing member, the first valve having a first closed state and a second open state, wherein in the first closed state the first valve blocks fluid communication between the first fluid chamber and the first nasal applicator member, and in the second open state the first valve facilitates fluid communication between the first fluid chamber and the first nasal applicator member;
a second valve in the first dispersing member, the second valve having a first closed state and a second open state, wherein in the first closed state the second valve blocks fluid communication between the second fluid chamber and the second nasal applicator member, and in the second open state the second valve facilitates fluid communication between the second fluid chamber and the second nasal applicator member;
a first fluid in the first fluid chamber, the first fluid being a first antidote;
a second fluid in the second fluid chamber, the second fluid being a second antidote;
a first nasal applicator member attached to the first dispersing member;
a second nasal applicator member attached to the second dispersing member; and
an actuator movably connected to the handle, the actuator being movable from a first actuator position to a second actuator position to move the first valve and the second valve to the second open state.

8. The intranasal delivery device of claim 7, wherein one of the first antidote and the second antidote is an antidote for a chemical agent.

9. The intranasal delivery device of claim 7, wherein one of the first antidote and the second antidote is an antidote for a biological agent.

10. The intranasal delivery device of claim 7, wherein in the second open state the valve facilitates fluid communication of a single dose of a first antidote from the first fluid chamber.

11. The intranasal delivery device of claim 7, wherein in the second open state the valve facilitates fluid communication of a single dose of a second antidote from the second fluid chamber.

12. An ergonomic intranasal delivery device, comprising:
a main body, the main body having disposed therein a syringe depressor joined to a first syringe and a second syringe, each of which extend out of a housing and into a nasal applicator housing, the nasal applicator housing extending from the main body at a proximal portion and being curved to a distal end;
a handle joined to the main body at an angle to the main body;
an actuator connected to the handle and elastically biased away from the handle;
a first fluid chamber in the nasal applicator housing, the first fluid chamber receiving the first syringe and in fluid communication with a first passageway;
a second fluid chamber in the nasal applicator housing, the second fluid chamber receiving the second syringe and in fluid communication with a second passageway;
a first nasal applicator member joined to the nasal applicator housing and in fluid communication with the first passageway;
a second nasal applicator member joined to the nasal applicator housing and in fluid communication with the second passageway;
a mechanical linkage between the actuator and the syringe depressor, wherein upon movement of the actuator toward the handle, the syringe depressor is urged in a linear motion, moving the first syringe and the second syringe into the first fluid chamber and the second fluid chamber, respectively.

13. An ergonomic intranasal delivery device of claim 12, further comprising a marker disposed on the nasal applicator housing.

14. An ergonomic intranasal delivery device of claim 12, further comprising a marker disposed on the nasal applicator housing, the marker comprising one of a dye and ink.

15. An ergonomic intranasal delivery device of claim 12, further comprising a first window providing a visual indication of the first fluid chamber.

16. An ergonomic intranasal delivery device of claim 12, further comprising a first window providing a visual indication of the first fluid chamber and a second window providing a visual indication of the second fluid chamber.

17. An ergonomic intranasal delivery device of claim 12, wherein the mechanical linkage comprises a pinion gear operationally engaged with a rack gear on the syringe depressor.

18. An ergonomic intranasal delivery device of claim 12, wherein the first fluid chamber comprises a first antidote.

19. An ergonomic intranasal delivery device of claim 12, wherein the second fluid chamber comprises a second antidote.

20. An ergonomic intranasal delivery device claim 12, further comprising a pliable covering the first nasal applicator member, the pliable covering defining an opening through which a first fluid dispensed from the first nasal applicator member can pass, the pliable covering further comprising an antimicrobial coating.
